(19) United States
(12) Patent Application Publication
(10) Pub. No.: US 2009/0099342 A1
(43) Pub. Date: Apr. 16, 2009

(54) Process for Preparing Composite Particles, Composite Particles Obtained, and Their Use in a Diagnostic Test

(75) Inventors: Sebastien Braconnot, Asniere sur Seine (FR); Abdelhamid Elaissari, Les Bastides des Chazelles (FR); Hanna Mouaziz, Le Mans (FR)

Correspondence Address: OLIFF & BERRIDGE, PLC, P.O. BOX 320850, ALEXANDRIA, VA, 22320-4850, US

(73) Assignees: BIOMERIEUX, MARCY L'ETOILE (FR); CENTRE NATIONAL DE LA RECHERCHE SCIENTIFIQUE (CNRS), PARIS (FR)

(21) Appl. No.: 12/084,576

(22) PCT Filed: Dec. 14, 2006
(86) PCT No.: PCT/FR2006/051355

§ 371 Date: May 6, 2008

(30) Foreign Application Priority Data
Dec. 15, 2005 (FR) 0553879

(51) Int. Cl. B01J 13/14; B32B 1/00; C07K 17/08

(52) U.S. Cl. 530/391.1; 264/4.7; 428/402.21

(57) ABSTRACT

The invention relates to a process for preparing composite particles by encapsulation of an organic-phase-depleted emulsion by polymerization, said emulsion consisting of droplets of inorganic emulsion comprising an organic phase and inorganic nanoparticles distributed in said organic phase, characterized in that the polymerization is carried out using, as the polymerization monomer, from 60% to 100% of at least one crosslinking agent and from 0% to 40% of at least one hydrophobic monomer, it being understood that at least 95% of the crosslinking agent(s) are hydrophobic. The invention further relates to the particles thus obtained, and their use in diagnostics.

The present invention relates to the field of composite particles. More particularly, the subject of the present invention is a process for preparing composite particles by encapsulation of an organic-phase-depleted emulsion by polymerization, said emulsion comprising droplets of inorganic emulsion comprising an organic phase and inorganic nanoparticles distributed in said organic phase, said process being characterized in that the polymerization is carried out using, as the polymerization monomer, from 60% to 100% of at least one crosslinking agent and from 0% to 40% of at least one hydrophobic monomer, it being understood that at least 95% of the crosslinking agent(s) are hydrophobic, and also to the novel composite particles thus obtained.

Encapsulation is a process used to obtain solid particles coated with at least one layer of polymer. Such a system is supposed to have properties different from the sum of the properties of the individual components, in particular better mechanical properties. Encapsulation processes have in particular been used in the field of the preparation of pigments, of inks, of plastics and of paints. One of the most important applications of encapsulated particles and pigments is found in the field of emulsion paints. However, when the inorganic particles obtained by encapsulation are magnetizable, this opens up specific pathways in the biology field, for example by virtue of the coupling of proteins or of antibodies to the encapsulated particles for use in diagnostic tests. Such particles are also used in biochemical separation processes. In general, the encapsulated particles are advantageous as a support, vector or carrier in the fields of biological engineering, diagnosis and pharmacy. To this effect, they have been used in medical diagnosis as a solid support for biological macromolecules.

Colloidal particles have several advantages compared with conventional solid supports, such as tubes and plates. In fact, they make it possible to have a large surface area for interactions, they diffuse in the volume, which increases the uptake kinetics, and they can be readily chemically modified by the introduction of functional groups capable of reacting with other natural, synthetic or biological molecules or macromolecules, such as antibodies or antibody fragments, proteins, polypeptides, polynucleotides, nucleic acids, nucleic acid fragments, enzymes, or chemical molecules such as catalysts, cage molecules, chelating agents, or even biological colloids such as bacteria, cells, viruses and parasites.

Among colloidal particles, magnetizable latices have attracted a great deal of interest in the analytical field and are used, for example, as a means for separating, concentrating and/or detecting analytes, such as antigens, antibodies, biochemical molecules, nucleic acids, bacteria, viruses, parasites, and the like.

It is possible to classify colloidal particles in three categories: small particles having a diameter of less than 50 nm, large particles having a diameter of strictly greater than 1000 nm, and intermediate particles having a diameter of between 50 and 1000 nm.

In order for the magnetic particles to be considered good candidates, in particular for a diagnostic application, they must meet certain criteria. A good content of inorganic filler (i.e. iron oxide), it is preferable for the magnetic filler to be distributed relatively homogeneously in the core and from one particle to the other. The magnetic filler should be completely encapsulated in order to avoid release of the magnetic filler. They should not aggregate irreversibly under the action of a magnetic field, which means that they may be readily and rapidly redispersed once the magnetic field has been eliminated.

Similarly, they should have a relatively low physical density in order to reduce the sedimentation phenomenon. Advantageously, they should have a narrow particle size distribution for homogeneous separation under the action of a magnetic field. The terms “monodisperse particles” or “isodisperse particles” are also used.

Thus, due to their size and their density, large magnetic particles in suspension in a liquid phase have a tendency to sediment rapidly.

Conversely, small magnetic particles have a tendency to remain in suspension due to their Brownien movement induced by thermal agitation, and are difficult (or even impossible) to separate with a magnet, in particular if the magnetic field applied is relatively weak. They are not therefore very suitable for the above uses.

There exists therefore an obvious advantage in producing composite particles having an intermediate size of between 50 and 1000 nm, which both overcome the abovementioned drawbacks and meet in particular the criteria established above. However, the invention is not limited to magnetizable composite particles, as described hereinafter.

Patent application EP-A-0 390 634 describes magnetizable composite microspheres of hydrophobic crosslinked vinylaromatic polymer having a diameter of the order of 50 to 10 000 nm and comprising a solid core constituted of magnetizable particles and a shell constituted of a hydrophobic copolymer derived from at least one hydrophobic vinylaromatic monomer and from at least one polyethylenically unsaturated emulsifying polymer soluble in the vinylaromatic monomer(s) and capable of crosslinking with said monomer(s). However, although they may meet the size requirement, the particles have the drawback that the magnetic filler, which is located inside the core, is not homogeneously distributed. Moreover, and as obviously emerges from the attached figures, the particles are not homogeneous in terms of size. This is therefore a collection of polydisperse particles which will have to be sorted by application of a magnetic field so as to retain only the particles of desired size.

Mention may also be made of Dynabead particles (trade name, Invitrogen). These particles are microspheres constituted of a porous core of polystyrene and of iron oxides. The oxides are deposited in the available pores by precipitation of the ferrous and ferric salts in a basic medium. In order to prevent release of the iron oxides, the encapsulation is carried out by introduction of a shell made of another polymer. The particles developed by this process have a diameter of 2.8 μm (M280 particles) and 4.5 μm (M450 particles), respectively. They are considered to be isodisperse particles, but, due to their large size, they have the abovementioned drawbacks, mainly the sedimentation phenomenon. Furthermore, their specific surface area is low.

Patent application WO 01/33223, filed by one of the applicants, describes composite nanospheres with a diameter of between 50 and 1000 nm, comprising an essentially liquid core constituted of an organic phase and of inorganic nanoparticles distributed within the organic phase, and a shell constituted of a hydrophilic polymer derived from the polymerization of at least one water-soluble monomer. These particles have a narrow size range, of the appropriate size, but have the drawback of not being stable in organic solvents.

Patent application WO 03/004559, also filed by one of the applicants, describes composite particles comprising a core made of a hydrophobic polymer forming the matrix and inorganic nanoparticles distributed in said matrix, said core being at least surrounded by an amphiphilic copolymer for the stabilization of the core and making it possible to introduce reactive functions at the surface of the particles. As above, these particles have a narrow size range, of the appropriate size, but have the drawback of not being stable in organic solvents.

According to the invention, against all expectations, novel composite particles are now provided which make it possible to overcome all the drawbacks mentioned above and which meet the following requirements:
spherical particles which have a very narrow size range, or even isodisperse,
particles having a homogeneous morphology tending toward a structure of the core/shell type (core surrounded by a polymer shell),
particles having an inorganic filler distributed homogeneously in the core,
particles of intermediate size, i.e. having a diameter of between 50 and 1000 nm, and preferably between 100 and 1000 nm,
functionalized or functionalizable particles,
their inorganic filler may be magnetic or magnetizable,
particles obtained by means of an original, simple and controllable method of synthesis,
particles stable in organic solvents, in particular polar solvents such as DMSO (dimethyl sulfoxide), DMF (dimethylformamide), acetonitrile and alcohols.

Thus, a subject of the present invention is a process for preparing composite particles by encapsulation of an organic-phase-depleted emulsion by polymerization, said emulsion comprising droplets of inorganic emulsion comprising an organic phase and inorganic nanoparticles distributed in said organic phase, said process being characterized in that the polymerization is carried out using, as the polymerization monomer, from 60% to 100% of at least one crosslinking agent and from 0% to 40% of at least one hydrophobic monomer, it being understood that at least 95% of the crosslinking agent(s) are hydrophobic.

The composite particles obtained by means of the process of the invention are novel and constitute another subject of the invention.

Finally, a subject of the invention is also the use of these novel composite particles in biomedical applications, and in particular diagnostic applications.

The inventors have therefore developed, for the first time, a novel process for preparing new generations of particles which are, against all expectations, very stable in the aqueous medium in which they are prepared, but also increasingly stable to very stable in the organic solvents into which they are transferred or transferable, the increase in stability depending on the increasing use of hydrophobic crosslinking agent. In addition, the process of the invention makes it possible to develop composite particles having a homogeneous morphology tending toward a structure of the core/shell type; the latter structure is conserved even when 100% of hydrophobic crosslinking agent is used as the polymerization monomer. The advantage of such a structure with a polymer shell is that the latter completely encapsulates the inorganic filler.

The characteristic of the process lies in the fact of using predominantly (at least 95% of 60% to 100%) at least one hydrophobic crosslinking agent as principal monomer in the presence of an organic-phase-depleted emulsion.

The term “organic-phase-depleted emulsion” is intended to mean an emulsion comprising an inorganic phase dispersed in an organic phase, said organic phase representing from 10 mg to 200 mg per gram of dry emulsion, preferably no more than 100 mg, and even more preferably no more than 50 mg per gram of dry emulsion.

As indicated above, the increase in the amount of hydrophobic crosslinking agent as the polymerization monomer in the process of the invention makes it possible to improve the stability in organic solvents of the composite particles obtained by means of said process, and to improve their structure with regard in particular to their homogeneity.

Thus, the process of the invention uses at least 70%, preferably at least 80%, more preferably at least 90% of at least one crosslinking agent. Use is even more preferably made of at least 95%, and even 100%, of at least one crosslinking agent, which constitutes an embodiment of the invention. 100% of hydrophobic crosslinking agent is also preferably used.

The process of the invention uses one or more crosslinking agent(s) in so far as the predominant (at least 95%) crosslinking agent(s) is (are) hydrophobic.

The term “hydrophobic crosslinking agent” is intended to mean any molecule or macromolecule having at least two vinyl bonds and exhibiting a water-solubility of less than 0.3 g/l.

Examples of a hydrophobic crosslinking agent include styrene derivative-based crosslinking agents, such as divinylbenzene and its derivatives, and any crosslinking agent having hydrophobic groups.

According to one embodiment of the invention, the process uses at least one crosslinking agent, and preferably at least one of said crosslinking agents is a styrene derivative-based crosslinking agent. Advantageously, the crosslinking agent is divinylbenzene.

According to another embodiment of the invention, the process uses a single crosslinking agent, preferably a styrene derivative-based crosslinking agent, and preferably divinylbenzene.

When the process uses at least two crosslinking agents, and preferably two crosslinking agents, at least one of the two is a fluorescent crosslinking agent or else a hydrophilic crosslinking agent.

The hydrophilic (nonhydrophobic) crosslinking agents comprise acrylamide derivatives such as methylenebisacrylamide and ethylene glycol dimethacrylate, and any crosslinking agent having water-soluble groups.

By way of fluorescent crosslinking agent, mention may be made of pyrene derivatives, derivatives of fluorescein bearing two methacrylate functions at its ends, such as fluorescein dimethacrylate (Polyfluor© 511), and naphthalene dimethacrylate. Such fluorescent crosslinking agents are available from Polysciences (US corporation).

According to a specific embodiment of the invention, the process uses no more than 5% of a hydrophilic crosslinking agent, preferably acrylamide-based, or of a fluorescent crosslinking agent.

The addition of a fluorescent crosslinking agent in the process of the invention makes it possible to obtain fluorescent particles in a single step, and their subsequent use in applications where labeling is appropriate, such as in biomedical applications such as diagnosis.

The addition of a hydrophilic crosslinking agent, in particular acrylamide-based, in the process of the invention makes it possible to obtain particles with a hydrophilic surface in a single step, and their subsequent use in applications where binding with a binding partner is appropriate, such as in biomedical applications such as diagnosis.

According to one embodiment of the invention, the process of the invention uses two crosslinking agents, one being a hydrophobic crosslinking agent, preferably divinylbenzene, in a predominant amount, and the other being a fluorescent crosslinking agent in a minor amount, preferably in a proportion of 1%, or else one being a styrene derivative-based crosslinking agent, preferably divinylbenzene, in a predominant amount, and the other being a water-soluble (acrylamide-based) crosslinking agent in a minor amount, preferably in a proportion of 5%.

The process of the invention also uses no more than 40% of at least one hydrophobic monomer.

The term “hydrophobic monomer” is intended to mean a monomer having a single vinyl bond and a water-solubility of less than 0.5 g/l.

Examples of such monomers comprise styrene monomers or derivatives, styrene being particularly preferred.

When, in the process of the invention, neither a fluorescent crosslinking agent nor a hydrophilic crosslinking agent is used, a fluorescent monomer may be used in a minor amount for labeling the particles.

Thus, according to one embodiment, the process of the invention uses from 0% to 30% and preferably from 0% to 35% of a hydrophobic monomer, in particular styrene monomer, and at most 10% and preferably 5% of a fluorescent hydrophobic monomer.

The fluorescent hydrophobic monomers are widely known to those skilled in the art.

By way of a fluorescent hydrophobic monomer, mention may be made of 2-naphthyl methacrylate (polyfluor 345), which is a fluorescent monomer used in a very low amount for functionalizing particles.

Thus, in the process of the invention, various monomer formulations may be used, it being understood that there is always a predominant amount of hydrophobic crosslinking agent and that there is never at the same time a minor amount (of no more than 10% and preferably 5%) either of fluorescent hydrophobic monomer or of fluorescent crosslinking agent or of hydrophilic crosslinking agent.

Nonlimiting examples of monomer formulations comprise:
60% to 99% of a hydrophobic crosslinking agent, in particular divinylbenzene, and 1% to 40% of a hydrophobic monomer, in particular styrene;
60% to 99.9% of a hydrophobic crosslinking agent, in particular divinylbenzene, 39.9% to 0% of a hydrophobic monomer, in particular styrene, and 0.1% to 10%, preferably 5%, either of fluorescent monomer or of fluorescent crosslinking agent or of hydrophilic crosslinking agent.

Examples of an organic phase in which the inorganic nanoparticles are distributed include phases comprising an aliphatic, cyclic or aromatic hydrocarbon. In particular, the hydrocarbon is chosen from alkanes, preferably containing at least five carbon atoms, such as pentane, hexane, heptane, octane, nonane, decane, undecane or dodecane, octane being particularly preferred.

The inorganic nanoparticles suitable for the purposes of the invention are any nanoparticles having a particle diameter of no more than 50 nm, preferably of no more than 10 nm, and even more preferably of at least 1 nm.

According to one embodiment, said nanoparticles are chosen from metal oxides of iron, of titanium, of cobalt, of zinc, of copper, of manganese, of nickel; magnetite; hematite; ferrites such as manganese ferrites, nickel, manganese-zinc; alloys of cobalt, nickel; zeolites; talc; clays such as bentonite and kaolin; alumina; silica; graphite; fluorescent crystals (such as, for example, CdSe); colloidal gold; and carbon black.

According to another embodiment, said inorganic nanoparticles are magnetic nanoparticles and they are chosen from metal oxides, and preferably iron oxides such as magnetite and maghemite.

Said inorganic nanoparticles are present in the emulsion in a proportion of from 40% to 80%, and preferably in a proportion of 60%.

The size of the emulsion is between 150 and 300 nm. It is preferably less than 250 nm, and even more preferably greater than 180 nm.

The preparation of the emulsion that is of use for the purposes of the invention may be carried out by any method known to those skilled in the art. An example of such a process is described by J. Bibette, 1993, J. Magn. Magn. Mater, 122:37 or in patent application WO 01/33223.

The process of encapsulation by polymerization of emulsion is widely known to those skilled in the art and is described, for example, in patent application WO 01/33223.

According to a preferred embodiment, the process for preparing composite particles of the invention comprises or consists of the steps consisting in:
(a) placing the emulsion in the presence of a surfactant;
(b) adding one or more hydrophobic crosslinking agent(s), and
(c) carrying out the polymerization.

The surfactants are widely known to those skilled in the art. According to one suitable embodiment, they are chosen from anionic surfactants such as amphiphilic polymers and sodium dodecyl sulfate, and nonionic surfactants such as triton X-405, the anionic surfactants such as amphiphilic polymers and sodium dodecyl sulfate being particularly preferred.

The amphiphilic polymers suitable for the purposes of the invention are, for example, described in patent application EP 892 020. Examples of such polymers are available from the company Coatex SA (France).

As indicated above, the polymerization principle is widely known to those skilled in the art. It is initiated by any initiating process known to those skilled in the art, such as by increasing the temperature so as to promote decomposition and/or in the presence of a radical initiator, or by photochemistry using radiation, such as UV radiation, or a laser beam or other sources of energy.

According to one embodiment of the invention, the polymerization is carried out in the presence of an initiator, preferably a water-soluble radical initiator.

Examples of water-soluble initiators comprise, for example, potassium persulfate and metabisulfite, potassium persulfate being particularly preferred.

The initiator is introduced either simultaneously with the introduction of the crosslinking agents, or prior to their introduction, or else after their introduction.

The polymerization is preferably carried out by increasing the temperature up to approximately 60 to approximately 90° C., preferably to approximately 70° C., in the presence of the polymerization initiator, it being understood that the polymerization conditions will be determined by those skilled in the art according to the initiation chosen.

The composite particles obtained by means of the process of the invention are novel, such that a subject of the invention is also the composite particles that may be obtained by means of the process described above.

The novel composite particles of the invention comprise a crosslinked polymer matrix in which inorganic nanoparticles are distributed.

The crosslinked polymer matrix is obtained using at least one crosslinking agent as defined above.

The inorganic nanoparticles come from the emulsion and represent from 40% to 80% by mass relative to the total mass of the emulsion in the dry state.

The composite particles of the invention in particular find applications in the field of paints, of inks, of plastics and, when they are functionalized, in various fields of biology, in particular for the separation of biological or biochemical molecules, for diagnostic tests, for cell sorting, for biological or biochemical capture, for analytical applications and also for supported chemistry.

According to one embodiment of the invention, the composite particles also have, at their surface, reactive functional groups capable of reacting with at least one ligand or one polymer.

The term “ligand” is intended to mean a biological molecule such as an antibody, an antibody fragment, a protein, a polypeptide, an enzyme, a polynucleotide, a probe, a primer or a nucleic acid fragment; a chemical molecule such as chemical polymers, medicinal substances, cage molecules, chelating agents or catalysts.

Examples of functional groups suitable for the purposes of the invention include carboxylic, amine, thiol, aldehyde, hydroxyl, tosyl and hydrazine groups, phenylboronic acid and activated esters.

These functional groups may be introduced by treatment of the surface of the particles, for example by chemical treatment such as hydrolysis or grafting, or else by the introduction of a functionalized crosslinking agent, or according to processes (chemical or physical) widely known to those skilled in the art.

According to another embodiment of the invention, the particles also have a binding agent specific for a substance capable of binding.

The expression “binding agent specific for a substance capable of binding” is intended to mean an intermediate compound which binds to a substance which is capable of binding to a ligand. By way of a specific binding agent/substance capable of binding pair, mention may be made of the pairs biotin/streptavidin, hapten/antibody, antigen/antibody, peptide/antibody, sugar/lectin, polynucleotide/polynucleotide complementary thereto.

According to a specific embodiment of the invention, the specific binding agent is streptavidin.

The particles of the invention may also in addition have, at their surface, a functionalized hydrophilic layer for subsequent binding to a ligand, which constitutes another embodiment of the invention.

By way of a functionalized hydrophilic layer, mention may be made of layers of natural or synthetic hydrophilic polymers, such as a layer of dextran, of chitosan, or of poly(N-alkylacrylamide), poly(N-alkylmethacrylamide), poly(acrylic acid) or poly(ethylene oxide) derivatives.

The hydrophilic layer may be applied to the particles of the invention by adsorption or covalent bonding according to processes widely known to those skilled in the art.

The particles thus modified and bound to a ligand are also novel.

Thus, a subject of the invention is also conjugates comprising a modified particle as defined above and a ligand, it being possible for said ligand to be bound to a substance capable of binding.

The conjugates also find applications in various fields of biology, in particular for the separation of biological or biochemical molecules, for diagnostic tests, for cell sorting, for the capture of bacteria, or for biological or biochemical capture.

Thus, another subject of the invention relates to the use of the particles or of the conjugates of the invention in an in vitro diagnostic test.

By way of an in vitro diagnostic test, mention may be made of the tests for assaying nucleic acids and derivatives, proteins and derivatives, and cells and derivatives.

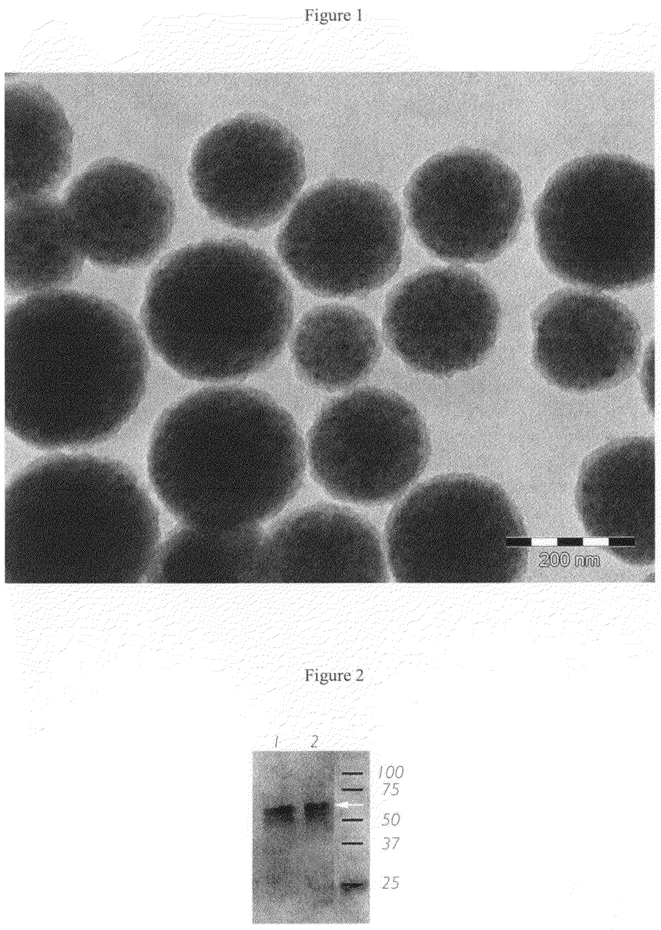

The invention will be understood more clearly from the following examples given only by way of nonlimiting illustration, and also from FIGS. 1 and 2 in which:

FIG. 1 is an electron micrograph of the particles of the invention prepared with 100% of hydrophobic crosslinking agent, and

FIG. 2 is a photograph of a Western blot demonstrating the capture of transmembrane protein (TM) derived from a cell lysat by the composite particles of the invention onto which anti-TM antibodies are grafted, lane 1 corresponding to particles to which 50 μg of antibodies were added, lane 2 corresponding to particles to which 150 μg of antibodies were added, the right-hand lane corresponding to the molecular weight marker lane.

### EXAMPLE 1

### Preparation of Particles of the Invention

The polymerizations are carried out in a glass reactor having a volume of 50 ml, equipped with a mechanical stirring system (half-moon-shaped Teflon anchor). This reactor is equipped with a condenser and has a jacket for the circulation of water from a thermostated bath.

The polymerizations are carried out in the presence of 50 ml of magnetic emulsion having a solids content of 4% (i.e. 2 g of emulsion) (Ademtech, emulsion prepared according to the protocol described in patent application WO 01/33223, said emulsion having from 10 to 250 mg of octane, from 20% to 30% of surfactants and from 70% to 80% of iron oxide, their particle diameter being from 180 to 270 nm). The emulsion is “washed” beforehand three times with an aqueous solution containing a surfactant (amphiphilic polymer Coatex M883 at 0.5 g/l prepared at pH 9), and then a last time with deionized, boiled and degassed water, before being finally introduced into the polymerization reactor. The emulsion “washing” operation consists in magnetically separating the droplets of the dispersant phase using a magnet and of replacing the continuous phase with a new one at equal volume. The reaction medium is subsequently maintained under an inert atmosphere (a few minutes under a regular stream of dinitrogen N₂).

The divinylbenzene (DVB) hydrophobic crosslinking agent (900 mg) is added to the magnetic emulsion with stirring so that it can readily diffuse in the droplets of emulsion, for one hour. Potassium persulfate (KPS) (18 mg) is added after the diffusion step and when the reaction medium is at the polymerization temperature. The polymerizations are carried out at a temperature of 70° C. for 20 hours.

The particles obtained are spherical and isodisperse, they have a homogeneous morphology of the core/shell type, they have an inorganic filler distributed homogeneously in the core, and they are of intermediate size, i.e. they have a diameter of between 50 and 1000 nm, as shown in FIG. 1, which is an electron micrograph taken after drying in the open air of a drop of the particles obtained.

Moreover, these particles have a magnetic or magnetizable inorganic filler, they are functionalized or functionalizable (see example 6 below) and they are obtained by means of an original, simple and controllable synthesis process.

### EXAMPLE 2

### Preparation of Particles of the Invention

The process is carried out as indicated in example 1 above, with the exception that 1500 mg of DVB were used.

### EXAMPLE 3

### Preparation of Particles of the Invention

The polymerizations are carried out in a glass reactor having a volume of 50 ml, equipped with a mechanical stirring system (half-moon-shaped Teflon anchor). This reactor is equipped with a condenser and has a jacket for the circulation of water from a thermostatic bath.

The polymerizations are carried out in the presence of 50 ml of magnetic emulsion having a solids content of 4% (i.e. 2 g of emulsion) (Ademtech, above). The emulsion is “washed” beforehand three times with an aqueous solution containing a surfactant (amphiphilic polymer Coatex M883 at 0.5 g/l prepared at pH 9), and then a last time with deionized, boiled and degassed water, before being finally introduced into the polymerization reactor. The emulsion “washing” operation consists in magnetically separating the droplets of the dispersant phase using a magnet and of replacing the continuous phase with a new one at equal volume. The reaction medium is subsequently maintained under an inert atmosphere (a few minutes under a regular stream of dinitrogen N₂).

The DVB crosslinking agent (900 mg) and the polyFluor 511 fluorescent crosslinking agent (1% by mass relative to the DVB, i.e. 9 mg) are added to the magnetic emulsion with stirring so that they can readily diffuse in the droplets of emulsion, for one hour. Potassium persulfate (KPS) (18 mg) is added after the diffusion step and when the reaction medium is at the polymerization temperature. The polymerizations are carried out at a temperature of 70° C. and for 20 hours.

### EXAMPLE 4

### Preparation of Particles of the Invention

The polymerizations are carried out in a glass reactor having a volume of 50 ml, equipped with a mechanical stirring system (half-moon-shaped Teflon anchor). This reactor is equipped with a condenser and has a jacket for the circulation of water from a thermostated bath.

The polymerizations are carried out in the presence of 50 ml of magnetic emulsion having a solids content of 4% (i.e. 2 g of emulsion) (Ademtech, above). The emulsion is “washed” beforehand three times with an aqueous solution containing a surfactant (amphiphilic polymer Coatex M883 at 0.5 gl/ prepared at pH 9), and then a last time with deionized, boiled and degassed water, before being finally introduced into the polymerization reactor. The emulsion “washing” operation consists in magnetically separating the droplets of the dispersant phase using a magnet and of replacing the continuous phase with a new one at equal volume. The reaction medium is subsequently maintained under an inert atmosphere (a few minutes under a regular stream of dinitrogen N₂).

The DVB crosslinking agent (900 mg) and a Polyfluor 345 fluorescent monomer (2% by mass relative to the DVB, i.e. 18 mg) are added to the magnetic emulsion with stirring so that they can readily diffuse in the droplets of emulsion, for one hour. Potassium persulfate (KPS) (18 mg) is added after the diffusion step and when the reaction medium is at the polymerization temperature. The polymerizations are carried out at a temperature of 70° C. and for 20 hours.

### EXAMPLE 5

### Preparation of Particles of the Invention

The polymerizations are carried out in a glass reactor having a volume of 50 ml, equipped with a mechanical stirring system (half-moon-shaped Teflon anchor). This reactor is equipped with a condenser and has a jacket for the circulation of water from a thermostated bath.

The polymerizations are carried out in the presence of 50 ml of magnetic emulsion having a solids content of 4% (i.e. 2 g of emulsion) (Ademtech, above). The emulsion is “washed” beforehand three times with an aqueous solution containing a surfactant (amphiphilic polymer Coatex M883 at 0.5 g/l prepared at pH 9), and then a last time with deionized, boiled and degassed water, before being finally introduced into the polymerization reactor. The emulsion “washing” operation consists in magnetically separating the droplets of the dispersant phase using a magnet and of replacing the continuous phase with a new one at equal volume. The reaction medium is subsequently maintained under an inert atmosphere (a few minutes under a regular stream of dinitrogen N₂).

The DVB crosslinking agent (900 mg) and the methylenebisacrylamide (MBA) water-soluble crosslinking agent (5% by mass relative to the DVB, i.e. 45 mg) are added to the magnetic emulsion with stirring so that they can readily diffuse in the droplets of emulsion, for one hour. Potassium persulfate (KPS) (18 mg) is added after the diffusion step and when the reaction medium is at the polymerization temperature. The polymerizations are carried out at a temperature of 70° C. and for 20 hours.

### EXAMPLE 6

### Functionalization of the Composite Particles Developed by Attachment of an Aminated Hydrophilic Polymer

6.1. Via Adsorption (Physical Process)

2 ml of the magnetic particles obtained in example 1 above are washed three times with a solution of Triton X-405 at 1 g/l. The washed particles are taken up in 1.9 ml of acetate buffer (0.002 M, pH 5.6). 100 μl of a solution of aminodextran (of varying concentration from 15 to 160 g/l) are added to the particles. The mixture is homogenized overnight. The particles are subsequently separated under the action of a magnetic field and washed with the acetate buffer (0.002 M, pH 5.6).

6.2. Via Chemical Reaction

2 ml of the magnetic particles obtained according to example 1 above are washed three times with a solution of Triton X-405 at 1 g/l. The washed particles are taken up in 1.9 ml of acetate buffer (0.002 M, pH 5.6). 100 μl of a solution of water-soluble carbodiimide, 1-(3-dimethylaminopropyl)-3-ethylcarbodiimide hydrochloride (EDAC), are added to the particulate dispersion, followed by incubation at ambient temperature for 40 minutes. The particles are washed three times with 1.9 ml of the acetate buffer (0.002 M, pH 5.6). 100 μl of a solution of aminodextran (of varying concentration from 15 to 160 g/l) are added to the particles. The mixture is homogenized overnight. The particles are subsequently separated under the action of a magnetic field and washed with the acetate buffer (0.002 M, pH 5.6). After overnight incubation, the magnetic particles are washed three times with the acetate buffer (0.002 M, pH 5.6).

### EXAMPLE 7

### Antigen Capture

7.1. Batch Grafting with an Organosoluble Carbodiimide

1 mg of the magnetic particles obtained in example 1 above (i.e. 25 μl, initial solids content of the particles: 4.07%) is used. After magnetic separation and elimination of the supernatant, the pellet is made up to 25 μl with 50 mM phosphate buffer pH 4.4+Tween 20 at 1%. 5 μl of dicyclohexylcarbodiimide (DCC) in DMSO are added at 50 mM (10 μl DCC+190 μl DMSO). 5 μl of N-hydroxysuccinimide (NHS) in DMSO at 1.12 M (i.e. 130 mg/ml) are added to the mixture, followed by 46.5 μl of antibody (i.e. 200 μg, initial antibody concentration: 4.3 mg/ml). The dispersion is made up to 500 μl with 50 mM phosphate buffer pH 4.4+Tween 20 at 1%. After incubation for 3 h at 37° C. (mixing in a thermomixer, 1000 rpm), magnetic separation and replacement of the supernatant with 10 mM phosphate buffer at pH 6.8 are carried out.

7.2. Two-Stage Grafting with an Organosoluble Carbodiimide:

1 mg of the magnetic particles obtained in example 1 above (i.e. 25 μl, initial solids content of the particles: 4.07%) is washed by magnetic separation and elimination of the supernatant, and redispersed in 25 μl of 50 mM phosphate buffer pH 4.4+Tween 20 at 1%. 5 μl of dicyclohexylcarbodiimide (DCC) in DMSO at 50 mM (10 μl DCC+190 μl DMSO) are then added, this operation being followed by the addition of 5 μl of N-hydroxysuccinimide (NHS) in DMSO at 1.12 M (i.e. 130 mg/ml). The dispersion is made up to 500 μl with 50 mM phosphate buffer pH 4.4+Tween 20 at 1%. The mixture is incubated at ambient temperature for 30 minutes on a mixer wheel. After magnetic separation and removal of the supernatant, 46.5 μl of antibody (i.e. 200 μg, initial antibody concentration: 4.3 mg/ml) are added and the dispersion is made up to 500 μl with 10 mM phosphate buffer, pH 6.8. After incubation for 3 h at 37° C. (stirring in a thermomixer, 1000 rpm), the magnetic particles are separated by application of a magnetic field and replacement of the supernatant with 10 mM phosphate buffer at pH 6.8.

7.3. Antibody Adsorption

1 mg of the magnetic particles obtained in example 1 above (i.e. 20 μl, initial solids content of the particles: 4.99%) is washed by magnetic separation, elimination of the supernatant, and redispersion in 20 μl of 10 mM phosphate buffer, pH 6.8. 46.5 μl of antibody (i.e. 200 μg, initial antibody concentration: 4.3 mg/ml) are then added, and the dispersion is made up to 500 μl with 10 mM phosphate buffer, pH 6.8. This operation is followed by an incubation step for 3 h at 37° C. (stirring with a thermomixer, 1000 rpm). After magnetic separation, the supernatant is replaced with 10 mM phosphate buffer at pH 6.8.

7.4. Grafting and Immunoprecipitation

22 μl of magnetic particles obtained in example 2 above, at 5.5% with respect to solids content, +98 μl of activation buffer (Ademtech): i.e. 120 μl of particles at 1%, are used. After magnetic separation and elimination of the supernatant, the particles are dispersed in 120 μl of activation buffer (operation repeated twice). 96 μl of N-(3-dimethylaminopropyl)-N′-ethylcarbodiimide hydrochloride (EDAC) at 4 mg/ml in activation buffer, i.e. a total volume of 216 μl, are then added. An incubation is carried out for 10 minutes at 40° C. (thermomixer stirring at 900 rpm), and is followed by the addition of 50 or 150 μg of anti-TM antibodies (6A2B2, bioMérieux, France) at 8.27 mg/ml per 54 μl of EDAC-activated particles. After incubation for 2 h at 37° C. (thermomixer stirring at 900 rpm), 15 μl of BSA at 2 mg/ml are added. After a final incubation for 30 minutes at 40° C. (thermomixer stirring at 900 rpm), the particles are separated magnetically and the supernatant is replaced with storage buffer (Ademtech).

30 μl of particles prepared as above, sensitized with the antibody, are added to 62 μl of cell lysat (lysat of TELCeb6 cells transfected with an envelope protein recognized by the anti-TM antibodies, J L. Blond et al., 2000, Journal of Virology, 74(7): 3321-3329) and 338 μl of lysis buffer (PBS-Triton X-100 at 0.05%). The mixture is stirred overnight (12 hours) at 4° C., which operation is followed by magnetic separation for two minutes and by washing with lysis buffer.

The proteins released (using the buffer tris, glycerol, 1% SDS and 5% beta-mercaptoethanol, for 5 minutes at 100° C.) from the magnetic pellet are analyzed by electrophoresis and Western blotting.

The results of the Western blotting are shown on the single figure, which is a photograph demonstrating the capture of transmembrane protein (TM) derived from the cell lysat by the particles of the invention onto which the anti-TM antibodies are grafted, either using 50 μg of antibodies (lane 1), or 150 μg (lane 2), the right-hand lane corresponding to the molecular weight marker lane.

The results demonstrate the capture of the proteins and therefore that the activity of the antibodies is not modified subsequent to its grafting. Moreover, the results also show that no difference is observed according to the amount of antibody used.

### EXAMPLE 8

### Demonstration of the Stability of the Particles of the Invention

Use was made of magnetic latex particles obtained in example 6.1, for which:
the size of the magnetic emulsion before polymerization is of the order of 220 nm,
after polymerization, the particle size is of the order of 267 nm and after functionalization with aminodextran, the average size is 325 nm, and the process was carried out as follows:
a) 1 ml of magnetic latex particles is separated under the action of a permanent magnet, the clear supernatant is eliminated and is immediately replaced with 1 ml of DMSO. After homogenization for a few minutes, the size of the particles is measured using light scattering (NanoZS from Malvern Instrument). The average size is 331 nm.
b) 1 ml of magnetic latex particles is separated under the action of a permanent magnet, the clear supernatant is eliminated and the pellet of particles is dried in the open air, i.e. at ambient temperature, for 48 h. The pellet is then redispersed in 1 ml of DMSO or in 1 ml of water using a vortex. The average particle size is between 330 nm and 380 nm.

The results above demonstrate that the particles of the invention are stable and redispersible in water and also in polar organic solvents.

1. A process for preparing composite particles having a diameter from 50 to 1000 nm, the process comprising:
encapsulating an organic-phase-depleted emulsion by polymerization, said emulsion consisting of droplets of inorganic emulsion comprising an organic phase and inorganic nanoparticles distributed in said organic phase, wherein the polymerization is carried out with, as the polymerization monomer, from 60% to 100% of at least one crosslinking agent of a plurality of crosslinking agents and from 0% to 40% of at least one hydrophobic monomer, at least 95% of the crosslinking agents being hydrophobic.

2. The process for preparing composite particles as claimed in claim 1, wherein the process is carried out with the at least 95% of the at least one crosslinking agent.

3. The process for preparing composite particles as claimed in claim 1, wherein the process is carried out with 100% of at least one hydrophobic crosslinking agent.

4. The process for preparing composite particles as claimed in claim 1, wherein the crosslinking agent is -a styrene derivative-based crosslinking agent.

5. The process for preparing composite particles as claimed in claim 4, wherein the crosslinking agent is divinylbenzene.

6. The process for preparing composite particles as claimed in claim 1, wherein the process is carried out with a single crosslinking agent.

7. The process for preparing composite particles as claimed in claim 1, wherein the process is carried out with 0% to 35% of a styrene hydrophobic monomer and no more than 5% of a fluorescent hydrophobic monomer.

8. The process for preparing composite particles as claimed in claim 1, wherein the process is carried out with no more than 5% of a fluorescent crosslinking agent or of an acrylamide-based crosslinking agent.

9. The process for preparing composite particles as claimed in claim 1, wherein the organic phase comprises an alkane.

10. The process for preparing composite particles as claimed in claim 9, wherein the alkane is octane.

11. The process for preparing composite particles as claimed in claim 1, wherein the inorganic nanoparticles are chosen from metal oxides of iron, of titanium, of cobalt, of zinc, of copper, of manganese, of nickel; magnetite; hematite; ferrites such as ferrites of manganese, nickel, manganese-zinc; alloys of cobalt, nickel; zeolites; talc; clays such as bentonite and kaolin; alumina, silica; graphite; fluorescent crystals; colloidal gold; and carbon black.

12. The process for preparing composite particles by encapsulation by polymerization of emulsion as claimed in claim 1, the process further comprising:
(a) placing the emulsion in the presence of a surfactant,
(b) adding one or more hydrophobic crosslinking agents as monomer, and
(c) carrying out the polymerization.

13. The process for preparing composite particles as claimed in claim 12, wherein the polymerization is carried out in the presence of an initiator.

14. The process for preparing composite particles as claimed in claim 13, wherein the initiator is a water-soluble radical initiator.

15. The process for preparing composite particles as claimed in claim 14, wherein the water-soluble radical initiator is potassium persulfate.

16. The process for preparing composite particles as claimed in claim 12, wherein the surfactant is an amphiphilic polymer or sodium dodecyl sulfate.

17. A composite particle having a diameter from 50 to 1000 nm, obtainable by means of the process as defined in claim 1.

18. The composite particle as claimed in claim 17, wherein at a surface of the composite particle, the composite particle has reactive functional groups capable of reacting with at least one ligand or one polymer.

19. The composite particle as claimed in claim 17, wherein the composite particle also has a binding agent specific for a substance capable of binding.

20. The composite particle as claimed in claim 19, wherein the specific binding agent is streptavidin.

21. The composite particle as claimed in claim 17, wherein, at a surface of the composite particle, the composite particle has a functionalized hydrophilic layer.

22. The composite particle as claimed in claim 21, wherein the functional hydrophilic layer is constituted of dextran.

23. A conjugate comprising a particle as defined in claim 18 and a ligand.

24. A conjugate comprising a particle as defined in claim 19 and a ligand bound to a substance capable of binding.

25. A process for a diagnostic test comprising:
a composite particle having a diameter from 50 to 1000 nm; or
a conjugate comprising the composite particle and a ligand,
the composite particle being obtainable by the process defined in claim 1.
